(19) United States
(12) Patent Application Publication
(10) Pub. No.: US 2007/0299365 A1
(43) Pub. Date: Dec. 27, 2007

(54) Sampling system for sample fluid

(76) Inventors: Irio Giuseppe Calasso, Arth (CH); Emad Sarofim, Hagendorn (CH)

Correspondence Address: ROCHE DIAGNOSTICS OPERATIONS INC., 9115 Hague Road, Indianapolis, IN, 46250-0457, US

(21) Appl. No.: 11/746,386

(22) Filed: May 9, 2007

Related U.S. Application Data
(63) Continuation of application No. PCT/EP05/11413, filed on Oct. 25, 2005.

(30) Foreign Application Priority Data
Nov. 9, 2004 (EP) 04026545.6

(51) Int. Cl. A61B 5/00

(52) U.S. Cl. 600/583

(57) ABSTRACT

The invention generally relates to a sampling system for sample liquid having a support, a lancing element arranged thereon and a semi-open channel for the capillary transport of the sample liquid from the lancing element to a collecting site on the support. A receiving structure is provided for receiving excess sample liquid escaping from the side of the channel.

### REFERENCE TO RELATED APPLICATIONS

The present application is a continuation of PCT Patent Application No. PCT/EP2005/011413, filed Oct. 25, 2005 which claims priority to European Patent Application No. 04026545.6 filed Nov. 9, 2004, which are hereby incorporated by reference in their entirety.

### TECHNICAL FIELD

The invention generally concerns a sampling system for sample liquid. In particular a microsampler for blood having a support, a lancing element and a semi-open channel for the capillary transport of the sample liquid from the lancing element to a collecting site on the support and a receiving structure for receiving excess sample liquid escaping from the side of the channel.

### BACKGROUND

Known prior art discloses a disposable lancing unit for removing small amounts of body fluid. The disposable lancing unit has a holding area that connects the proximal end of an elongate capillary structure with at least one capillary channel for transporting body fluid and the distal end of the capillary structure is suitable for piercing skin where the at least one capillary channel is open to the outside at least over a part of its length. The open capillary structure is not only advantageous with regard to manufacturing but also improves the uptake of samples from small puncture wounds that are desirable in order to reduce the puncture pain. As a result of the capillary-driven flow of blood to the detection zone or collecting site, it is possible to integrate the sample removal, the transport and the detection for example of blood sugar into one system. In order to improve the measurement it has already been proposed to load two-dimensionally-extended detection areas via the main transport capillary. However, even in this case there is the problem that when there is an excess blood flow at the removing site compared to the transport capacity of the channel, an undesired escape of blood on the transport path can lead to a contamination of the system with potentially dangerous biofluid. Moreover, the functionality of the removal system is impaired by such an undirected blood flow.

With this as a starting point the object of the invention is to overcome the disadvantages occurring in the prior art and to reduce the risk of contamination.

### SUMMARY

The idea behind the invention is to specifically divert any excess liquid on the transport path. Accordingly, the invention proposes a receiving structure for excess sample liquid escaping from the side of the channel. In this manner the excess sample liquid is functionally retained on the support so that no undefined contamination occurs. This is especially important when the capillarity of the channel or the amount of liquid flowing in it is small as is the case for very small capillaries or capillaries having a small aspect ratio. According to the invention a valve function is also created which automatically becomes effective when a critical flow amount is reached.

The receiving structure is effective as a capillary for the sample liquid at least in an entry region near to the channel such that an automatic suction effect is achieved.

In one of the embodiments, the capillary transport capacity for the sample liquid is larger in the direction of the channel than in the branch direction of the receiving structure. The set differences in capillarity do not prevent the liquid from continuing to flow in the direction of the collecting site.

The receiving structure is arranged on the support at a middle section of the channel downstream of the lancing element and upstream of the collecting site with respect to the flow direction. If the lancing element sticks out as a tip on a shaft member of the support, it is advantageous when the receiving structure is arranged in the area of the shaft member.

The receiving structure is formed by a cover element arranged on the support while keeping free a capillary gap towards the channel. As a result the amount of liquid drown into the capillary gap is at the same time screened from the outside. This should ensure that the capillary gap has a lower capillary attraction for the sample liquid than the adjoining section of channel so that the liquid can flow back into the channel.

In yet another embodiment, the receiving structure is formed by at least one semi-open overflow capillary which is arranged next to the side of the channel on the support part. It is expedient when two overflow capillaries are arranged symmetrically to one another on both sides of the channel. In order to increase the volumetric capacity, the at least one overflow capillary can have at least one branch.

In order to not unnecessarily influence the regular sample transport, the channel is separated from the at least one overflow capillary by a side wall, where excess sample liquid overflows into the overflow capillary over the side wall. Alternatively it is also possible that the channel is fluidically connected to the at least one overflow capillary by a capillary branch. In this connection it should be ensured that the branch has a smaller flow cross-section than the adjoining overflow capillary.

Another improvement is achieved by means of the fact that the receiving structure at the same time forms a reservoir for refilling the channel.

For the manufacturing process, the lancing element is formed on a flat shaped part being the support and when the channel has a linear groove shape. The support and the lancing element are formed as one piece from a flat material by photochemical mask etching. Alternatively, they could be formed by two separate pieces that are joined together.

Another aspect of the invention concerns a blood analyzer having at least one sampling system.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a microsampler for blood in a top-view;

FIG. 2a and b show a section along the line 2-2 of FIG. 1 with various blood flows; and

FIGS. 3 and 4 show further embodiments in a top-view.

Skilled artisans appreciate that elements in the figures are illustrated for simplicity and clarity and have not necessarily been drawn to scale. For example, the dimensions of some of the elements in the figure may be exaggerated relative to other elements to help improve understanding of the embodiment(s) of the present invention.

In order that the invention may be more readily understood, reference is made to the following examples, which are intended to illustrate the invention, but not limit the scope thereof.

### DETAILED DESCRIPTION

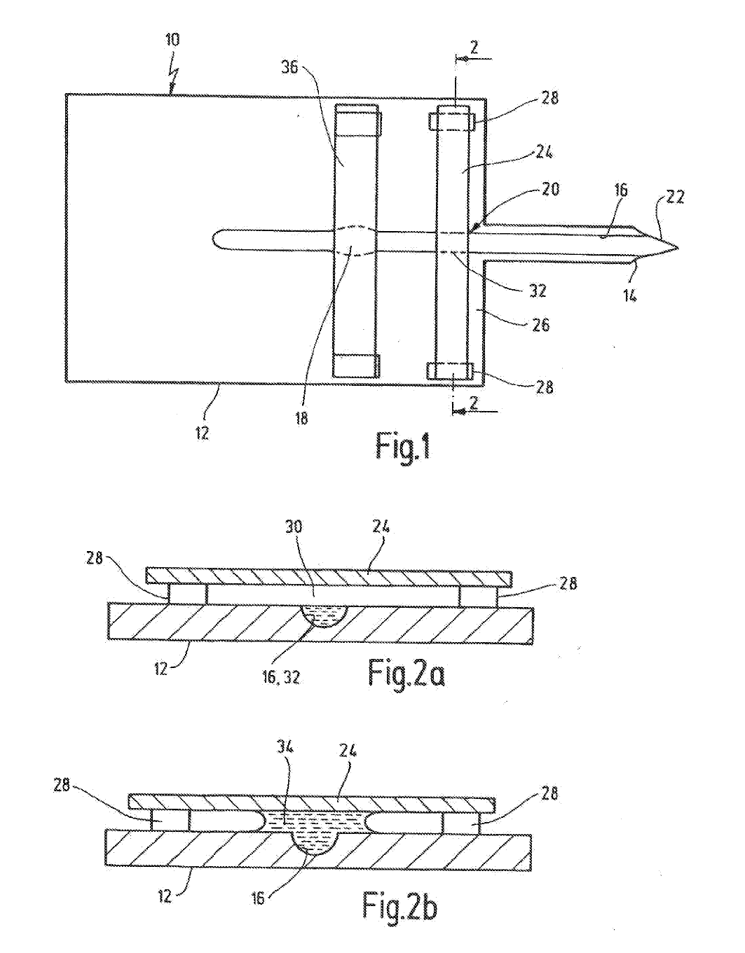

Referring to FIG. 1, a microsampler is shown and represented by reference number 10. The microsampler 10 comprises a support 12, a lancing element 14 moulded thereon, a semi-open channel 16 for transporting blood from the lancing element 14 to a collecting site 18 and a receiving structure 20 for receiving excess blood that may escape from the side of the channel 16.

The support 12 shown in FIG. 1 with the lancing element 14 formed thereon is formed in a known manner from a thin sheet of stainless steel by photochemical mask etching. The needle tip 22 of the lancing element 14 can for example be inserted into the fingerpad of a test subject to collect a microscopic amount (nano to microtiter) and the blood is automatically transported to the collecting site 18 by the capillarity of the channel 16. In order to avoid accidental contamination, the holding structure 20 is designed for a defined uptake of excess blood.

The receiving structure 20 is formed by a cover element 24. The cover element 24 spans the channel 16 in a shaft region 26 of the support 12 which adjoins the lancing element 14, it is held at a small distance from the support 12 by the spacer 28 so that a capillary gap 30 remains free above the underlying section of channel 32. The capillary gap 30 has a lower capillary attraction for blood than the channel section 32. In this manner the blood flowing in the channel 16 is not prevented from reaching the collecting site 18. Only when the amount of blood flowing from the puncture site is larger than the holding capacity of the channel 16, is the excess blood 34 retained in the capillary gap 30 in a spatially defined manner under the cover element 24. Optionally the excess 34 can also be used to refill the channel 16 when the amount flowing out of the puncture site subsequently decreases.

The channel 16 which is semi-open and groove-shaped over its length extends linearly form the needle tip 22 to beyond the collecting site 18. A detection element 36 is in fluidic contact with the blood that collects at the laterally widened collecting or target site 18. The detection element 36 responds to an analyte, for example glucose in blood, so that a quantitative detection can be carried out by a detection unit that is not shown.

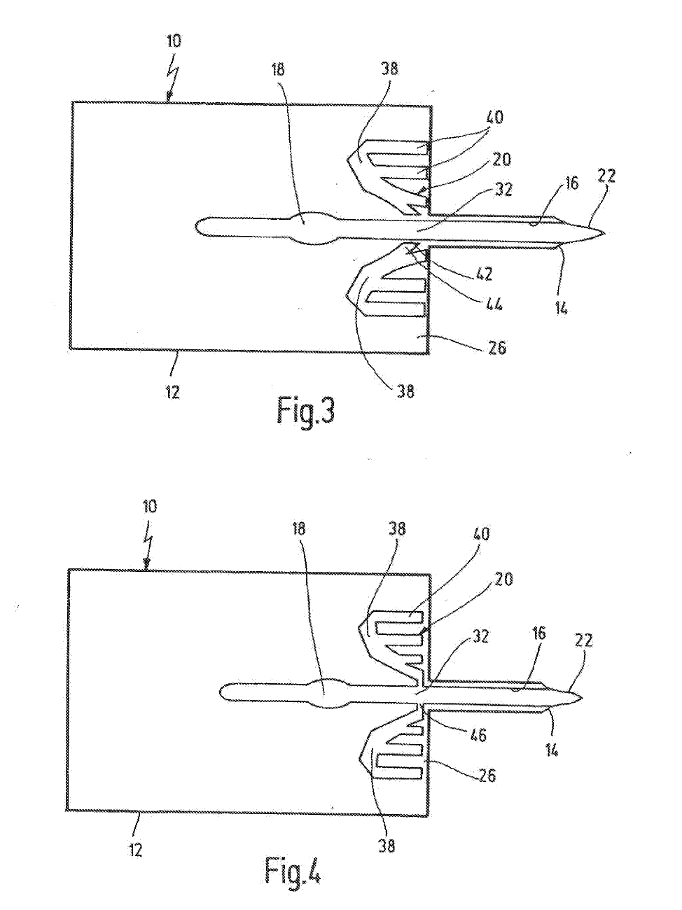

In the embodiments shown in FIGS. 3 and 4 overflow capillaries 38 form the receiving structure 20 for excess blood instead of the cover element 24. The overflow capillaries 38 are in each case arranged symmetrically to one another in pairs on both sides of the channel in the shaft area 26 of the support part 12. Branches 40 are provided in order to increase the holding capacity.

In the embodiment according to FIG. 3 the overflow capillaries 38 are separated by a side wall 42 from the adjoining section of channel 32 so that its capillarity is not weakened. Also in this case excess blood only passes over the side wall 42 into the overflow capillaries 38 when the inflow is excessive. The excess is automatically taken up by the capillary activity of at least the channel-side entry area 44 of the overflow capillaries 38.

In the embodiment shown in FIG. 4 the overflow capillaries 38 are each directly connected to the channel 16 via a branch 46. In this case it is advantageous when the branch 46 forms a very short and thin connecting capillary. Also in this case it should be ensured that the capillary transport capacity in the direction of the channel 16 is larger than in the branch direction.

The microsamplers 10 can be used in a near-patient environment as so-called disposables or single-use products in portable blood sugar measuring instruments in order to hygienically carry out correct blood sugar determinations in the daily routine with little handling and less puncture pain.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the invention in detail and by reference to specific embodiments thereof, it will be apparent that modification and variations are possible without departing from the scope of the invention defined in the appended claims. More specifically although some aspects of the present invention are identified herein as preferred or particularly advantageous, it is contemplated that the present invention is not necessarily limited to these preferred aspects of the invention.

What is claimed is:

1. A microsampler for sampling a body liquid, the microsampler comprising:
a support;
a lancing element arranged on the support;
a semi-open channel for the capillary transport of the sample liquid from the lancing element to a collecting site;
a receiving structure arranged on the support for retaining excess body liquid escaping from the side of the channel.

2. The microsampler of claim 1, wherein the receiving structure is effective as a capillary for the body liquid in an entry area on the channel side.

3. The microsampler of claim 1, wherein the capillary transport capacity for the body liquid is larger in the direction of the channel than in the branch direction of the receiving structure.

4. The microsampler of claim 1, wherein the receiving structure is arranged on the support at a section of the channel downstream of the lancing element and upstream of the collecting site with respect to the flow direction.

5. The microsampler of claim 1, wherein the lancing element sticks out as a tip on a shaft member of the support and that the receiving structure is arranged in the area of the shaft member.

6. The microsampler of claim 1, wherein the receiving structure is formed by a cover element arranged on the support while keeping free a capillary gap towards the channel.

7. The microsampler of claim 6, wherein the capillary gap has a lower capillary attraction for the body liquid than the adjoining section of channel.

8. The microsampler of claim 1, wherein the receiving structure is formed by at least one semi-open overflow capillary which is arranged next to the side of the channel on the support.

9. The microsampler of claim 8, wherein two overflow capillaries are arranged symmetrically to one another on both sides of the channel.

10. The microsampler of claim 8, wherein at least one overflow capillary can have at least one branch.

11. The microsampler of claim 10, wherein the at least one branch has a smaller flow cross-section than the adjoining overflow capillary.

12. The microsampler of claim 8, wherein the channel is separated from the at least one overflow capillary by a side wall, where excess body liquid overflows into the overflow capillary over the side wall.

13. The microsampler of claim 12, the channel is fluidically connected to the at least one overflow capillary by a capillary branch.

14. The microsampler of claim 1, wherein the receiving structure at the same time forms a reservoir for refilling the channel.

15. The microsampler of claim 1, wherein the lancing element is formed on a flat shaped part being the support and that the channel has a linear grooved shape.

16. The microsampler of claim 1, wherein the support and the lancing element are formed as one piece from a flat material by photochemical mask etching.
